SEQUENCE LISTING (14 sequences)

SEQ ID NO: 1 - DNA, 30 bp, Staphylococcus epidermidis
cttaaccatggctgaaacggacaatatcgt

SEQ ID NO: 2 - DNA, 24 bp, Staphylococcus epidermidis
tttattgcttattgcttttactgc

SEQ ID NO: 3 - DNA, 30 bp, Staphylococcus epidermidis
cttaaccatggatagtgacaaacatgaaat

SEQ ID NO: 4 - DNA, 21 bp, Staphylococcus epidermidis
agttttatcattagcccctac

SEQ ID NO: 5 - DNA, 33 bp, Staphylococcus epidermidis
cttaaccatggaagaattcgaaagagtaactac

SEQ ID NO: 6 - DNA, 24 bp, Staphylococcus epidermidis
ttcagtatcatttttaacatctgt

SEQ ID NO: 7 - DNA, 31 bp, Staphylococcus epidermidis
cttaaccatggatgaaaatactgcaaaccaa

SEQ ID NO: 8 - DNA, 26 bp, Staphylococcus epidermidis
tttatcttgtacatcattgttattat

SEQ ID NO: 9 - DNA, 35 bp, Staphylococcus epidermidis
cttaaccatgggctgattatgtaaatgactcaaat

SEQ ID NO: 10 - DNA, 21 bp, Staphylococcus epidermidis
agcttttgttgtttgagcttc

SEQ ID NO: 11 - DNA, 34 bp, Staphylococcus epidermidis
cttaaccatggattcagaaagtacatcatcaaat

SEQ ID NO: 12 - DNA, 21 bp, Staphylococcus epidermidis
tgagatattaccatcgtcgtc

SEQ ID NO: 13 - DNA, 31 bp, Staphylococcus epidermidis
cttaaccatggccgaagtaacatctcatgat

SEQ ID NO: 14 - DNA, 25 bp, Staphylococcus epidermidis
catatcagatgaactttgttgtggt

(19) United States
(12) Patent Application Publication
(10) Pub. No.: US 2009/0214584 A1
(43) Pub. Date: Aug. 27, 2009

(54) Characterization of novel lpxtg-containing proteins of staphylococcus epidermidis

(75) Inventors: Bengt Guss, Uppsala (SE); Jan Ingmar Flock, Bromma (SE)

Correspondence Address: DINSMORE & SHOHL LLP, 1900 CHEMED CENTER, 255 EAST FIFTH STREET, CINCINNATI, OH, 45202, US

(73) Assignee: Biostapro AB

(21) Appl. No.: 11/921,405

(22) PCT Filed: May 30, 2006
(86) PCT No.: PCT/IB2006/003867

§ 371 Date: Nov. 30, 2007

Related U.S. Application Data
(60) Provisional application No. 60/685,964, filed on May 31, 2005.

(51) Int. Cl. A61K 39/085; C07K 14/31; C07K 16/12; G01N 33/566; A61P 31/04

(52) U.S. Cl. 424/190.1; 530/350; 530/387.9; 436/501

(57) ABSTRACT

The present invention is directed toward isolated Staphylococcus epidermidis surface (Ses) A-F proteins, monoclonal and polyclonal antibodies directed against SesA-F proteins, vaccines comprising SesA-F proteins and diagnostic devices. The present invention is further directed to method of diagnosing an individual with Staphylococcus epidermidis comprising obtaining a sample from the individual and analyzing the sample for a Staphylococcus epidermidis surface (Ses) protein, wherein the Ses protein comprises SesA protein, SesB protein, SesC protein, SesD protein, SesE protein, SesF protein or a combination thereof.

### FIELD OF THE INVENTION

The present invention is directed toward isolated Staphylococcus epidermidis surface (Ses) A-F proteins, monoclonal and polyclonal antibodies directed against SesA-F proteins, and vaccines comprising SesA-F proteins. The present invention is also directed to a method of diagnosing an individual with an infection caused by Staphylococcus epidermidis. The method comprises obtaining a sample from the individual and analyzing the sample for a Staphylococcus epidermidis surface (Ses) protein, wherein the Ses protein comprises SesA protein, SesB protein, SesC protein, SesD protein, SesE protein, SesF protein or a combination thereof. The diagnostic methods may also be based on detection of antibodies from an individual against at least one Ses protein.

### BACKGROUND OF THE INVENTION

Staphylococcus epidermidis is a major nosocomial pathogen. It is a common etiologic agent in neonatal septicemia, in peritonitis, and in foreign body associated infections. In patients, proteins such as albumin, fibrinogen, fibronectin and von Willebrand factor rapidly cover implants. S. epidermidis possesses an unknown number of surface localized proteins that contribute to specific adhesion to these molecules. Surface proteins are also involved in adhesion to host tissue, biofilm formation and in other ways contributing to pathogenicity. These surface proteins are putative targets for antibody based prophylaxis and therapy. The prospect of using passive immunization or vaccination against infections caused by S. epidermidis relies on the assumption that target structures on the surface can be identified, against which protective antibodies can be directed.

Cell wall associated surface proteins have common motifs that enables them to be correctly sorted and attached to peptidoglycan. A signal peptide in the N-terminal is required for sec-dependent secretion and a C-terminal cell-wall sorting signal is needed for sortase-mediated attachment to peptidoglycan. The C-terminal sequence is comprised of a LPXTG motif for cleavage and attachment to peptidoglycan by sortase, followed by a membrane spanning hydrophobic stretch and positively charged residues in the very end of the C-terminal. The C-terminal motif is called the cell-wall sortase signal (Cws).

The so far best characterized cell wall associated protein of S. epidermidis, Fbe, is a 119-kDa fibrinogen binding protein present in most tested strains of S. epidermidis. Fbe has also been named SdrG. It is structurally related to other staphylococcal surface located proteins, such as ClFA of S. aureus. S. epidermidis interaction with peripheral venous catheters from patients, which spontaneously become coated with fibrinogen, is blocked by anti-Fbe antibodies. Fbe is expressed on the surface of S. epidermidis during infection, indicated by an increase of anti-Fbe titers in sera from patients infected with S. epidermidis. Anti-Fbe antibodies also protect from experimental infection in mice and enhance phagocytosis by macrophages.

Other known Cws proteins are, Aap, a cell division protein belonging to the FtsW/RodA/SpoVE family, Bhp, SdrF and possibly SdrH. S. epidermidis also possess a number of cell wall associated proteins that lack the Cws; the autolysins Aas and AtlE, the GhD lipase, the proteinase Ecp, the lantibiotic Pep1, the transferrin binding protein Tnp and Ssp1 and 2.

Macrophages are of vital importance in suppressing S. epidermidis infections. Phagocytic activity of macrophages is greatly enhanced if specific antibodies recognize and bind to the pathogen.

### SUMMARY OF THE INVENTION

The present invention is directed toward isolated Staphylococcus epidermidis surface (Ses) A-F proteins. The present invention is also directed to monoclonal and polyclonal antibodies directed against SesA-F proteins. The present invention is also directed to vaccines comprising SesA-F proteins. The present invention is further directed to a diagnostic device comprising at least one reagent operable to bind a Staphylococcus epidermidis surface (Ses) A-F proteins.

The present invention is also directed to a method of diagnosing an individual with an infection caused by Staphylococcus epidermidis. The method comprises obtaining a sample from the individual and analyzing the sample for a Staphylococcus epidermidis surface (Ses) protein. The Ses protein comprises SesA protein, SesB protein, SesC protein, SesD protein, SesE protein, SesF protein or a combination thereof.

The present invention is further directed to a method of diagnosing an individual with an infection caused by Staphylococcus epidermidis. The method comprises obtaining a sample of immunoglobulin from the individual and analyzing the sample for immunological reactivity against Staphylococcus epidermidis surface (Ses) protein. The Ses protein comprises SesA protein, SesB protein, SesC protein, SesD protein, SesE protein, SesF protein or a combination thereof. The diagnostic methods may also be based on detection of antibodies from an individual against at least one Ses protein.

### DETAILED DESCRIPTION OF THE FIGURES

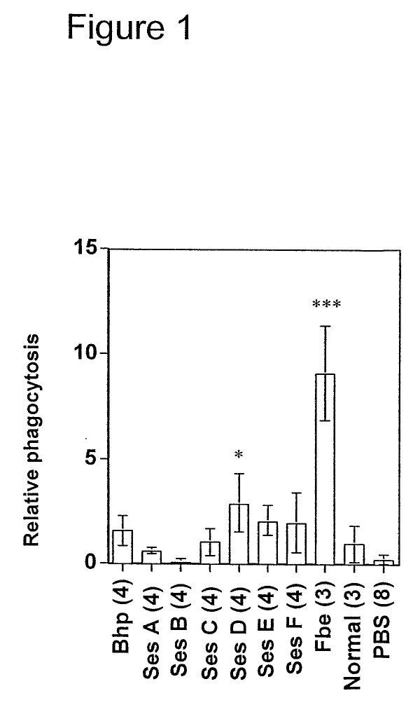

FIG. 1: Phagocytosis of opsonised S. epidermidis by TEP-1 macrophages. Relative phagocytosis (as compared to normal serum in each experiment) of S. epidermidis RP62A opsonised with antibodies directed against individual cell wall surface associated proteins. Mean values and SD are shown. The number of individual trials for each assay is noted. Bacteria to macrophage ratio is 1. ***p<0.001, *p<0.05.

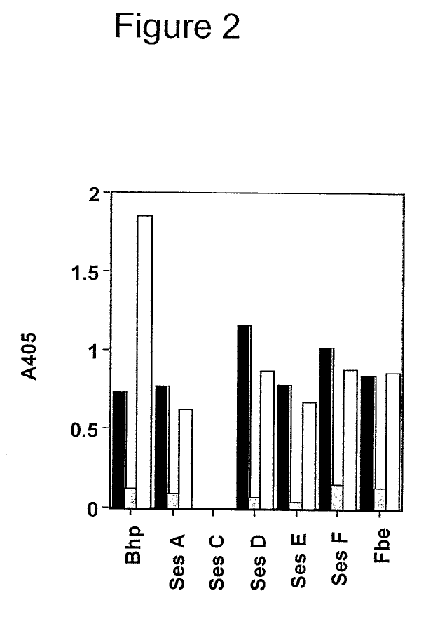

FIG. 2: Antibodies against Ses proteins in IgG pools. Three different IgG pools are used (black, grey or white bars) in ELISA. IgG at 15 μg/ml is used to detect the Ses proteins in ELISA. Background reactivity against BSA has been subtracted. The experiment shown is a typical example of several.

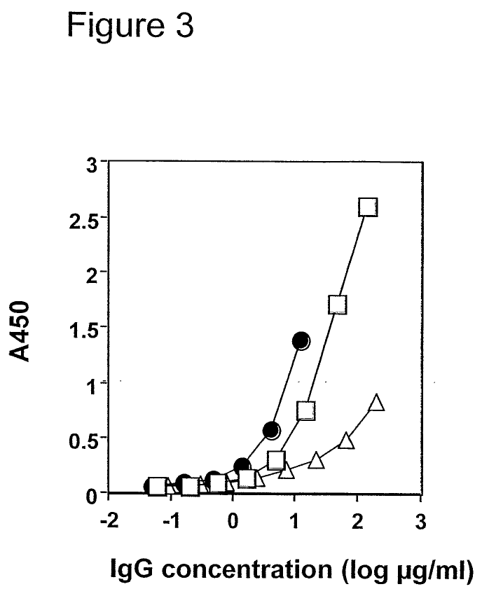

FIG. 3: Titration in ELISA of antibodies against Fbe. Anti-Fbe IgG in pool 1 (open squares), enriched anti-Fbe (filled circles) and effluent devoid of anti-Fbe antibodies (open triangles) are measured in ELISA.

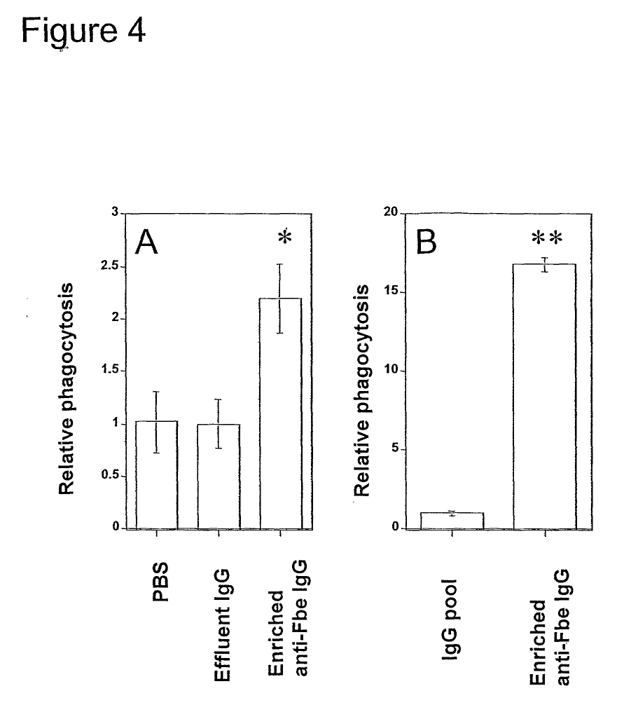

FIG. 4: Opsonic activity of anti Fbe antibodies. S. epidermidis is opsonised for one hour with IgG (at 10 μg/ml in all cases) and then given to macrophages. The bacterial uptake into macrophages is determined after 90 minutes. Experiment A shows uptake using antibodies enriched for anti-Fbe antibodies, IgG in the effluent devoid of anti-Fbe antibodies and PBS as negative control. Values are given as relative values with the effluent IgG set at 1.0. The ratio of bacteria added to macrophage is 10 in this experiment. Experiment B shows enriched antibodies against Fbe and IgG pool 1, from which enriched anti Fbe IgG is derived. Values are given as relative values with the IgG pool set at 1.0. The ratio of bacteria added to macrophage is 1 in this experiment. *p<0.05 and **p<0.01

### DETAILED DESCRIPTION OF THE INVENTION

The inventors have identified 7 novel Cws proteins. Specifically, six Cws are confirmed to be localized on the surface of RP62A, with varying prevalence in S. epidermidis isolates. These novel proteins are specifically called Staphylococcus epidermidis surface (Ses) proteins. Macrophage phagocytosis of S. epidermidis RP62A is enhanced by anti-Fbe antibodies, and by antibodies directed against one of the novel Cws proteins, here called SesD. The other Cws are far less effective in eliciting antibodies stimulating phagocytosis.

The inventors have evaluated and compared 8 cell wall surface anchored proteins from S. epidermidis as potential targets for opsonic antibodies. Fbe is a previously characterized fibrinogen binding protein included for comparison. In previous studies the inventors have demonstrated that Fbe is a target for opsonic antibodies. The Fc portion of IgG is recognized by macrophages that ingest the opsonised bacteria to a higher extent compared to unopsonised bacteria. The increased uptake leads to increased and faster killing, thus improving clearance of the bacteria. Consequently, antibodies against Fbe are effective in clearing infection in an experimental mouse model.

In the search for novel targets for antibody treatment of S. epidermidis infections, the inventors searched the genome of strain RP62A for proteins possibly located at the bacterial surface. The genome is searched for the cell-wall sorting signal (CWS) and signal peptide previously reported to be required for sortase mediated cell wall anchoring. Ten proteins are found. Previously known proteins are Fbe, AtlE, a protein belonging to the FtsW/RodA/SpoVE family, and the protein Blip similar to S. aureus Biofilm associated protein, Bap. Six novel proteins are thus identified here called Ses A-F. Recently, these proteins have been identified as the “cell wall surface anchored protein family”. One additional protein that is not identified in the inventors' primary search is also included in the family (NCBI accession No:AAW54644).

The number of identified sequences with the CWS identified in S. epidermidis RP62A is thus 11, which is in line with previously reported data. S. epidermidis strain 12228 harbors 9 proteins with LPXTG motifs; Fbe/SdrG, Aap, SdrF, FmtB, homologues to SesB, C and E, and 2 proteins not identified as Cws in this study. This could be compared to S. aureus, in which 21 or 22 LPXTG containing proteins are identified. Group A streptococci contain 28 Cws, Streptococcus pneumoniae 19 and Enterococcus fecalis harbors 41. S. epidermidis is known to have proteins expressed on its surface that lack the CWS, but in this study, the inventors have focused on the CWS containing sequences.

From the PCR on 20 strains of S. epidermidis the inventors can see that the genes for Bhp is present in only 3 of the 19 clinical isolates tested. SesD is present in 17 of 19 strains. All other except SesA is present in all the 19 tested strains. SesA is present in 3/9 skin-isolates, and in 6/10 infection isolates. Whether this indicate a higher prevalence of SesA in infection isolates needs to be determined in a larger sample of clinical isolates.

In silico analysis also reveals the presence of several of the genes in the strains ATCC 12228 and SR1. Homology searches resulted in a very limited number of hits on identity with other known proteins or genomes. A general conclusion is that the open reading frames for the Cws containing sequences described in this study are common in S. epidermidis, but are not generally found in other published nucleotide or protein sequences.

The first prerequisite for antibody-mediated opsonisation is that the target is exposed at the surface of the bacteria at the growth conditions used. The opsonic activity of specific antibodies directed against each protein is tested in a macrophage phagocytosis assay. Mock-preopsonisation is performed with PBS. In comparison with PBS, all sera, including normal sera, enhanced phagocytosis by macrophages. As previously described, immunization with Fbe resulted in opsonic antibodies, increasing phagocytosis compared to normal serum. The inventors have here shown that also antibodies against SesD have opsonic capacity. Immunization with the other novel proteins or Bhp did not result in enhanced phagocytosis. If this is due to poor expression in the specific strain RP62A or generation of poorly opsonic antibodies is not clear.

The expression of the Ses proteins, except SesF, in broth culture is demonstrated by western blot analysis. The absence in broth culture of SesF might be due to expression below detection level or proteolytic instability. However, expression of the Ses proteins in vivo is of far greater importance for therapeutic antibodies. The inventors have shown here that human IgG pools contain antibodies against all Ses proteins except SesC, but to varying extents. This strongly implies that the Ses proteins are expressed in vivo during colonization or infection; some donors might have undergone recent S. epidermidis infections resulting in anti Ses antibodies.

The inventors have previously shown that anti-Fbe antibodies resulting from immunization of sheep or rats have opsonic activity. The inventors have now also demonstrated that naturally occurring antibodies, such as in IVIG preparations, against Fbe, although at low titer, display opsonic activity if enriched by affinity chromatography.

Enrichment of specific antibodies may enhance the opsonic activity of antibodies directed also against the other novel proteins, but is not studied here. So far, opsonisation of S. epidermidis with Fbe antibodies has resulted in increased macrophage phagocytosis in all strains tested, and is a strong candidate for antibody-mediated therapy against S. epidermidis. Also antibodies against SesD are shown is in this study to be a novel target for antibody mediated therapy.

In summary, at least 11 LPXTG-anchored surface proteins are encoded for in S. epidermidis Strain RP62A. The prevalence in clinical isolates is high among the newly identified open reading frames (17-19/19), except for Bhp (3/19) and SesA (9/19).

### EXAMPLES

### Materials and Methods

### Prediction of Novel LPXTG Proteins.

A survey of genes encoding potential cell wall associated proteins in S. epidermidis is performed with the finished genome sequence of strain RP62A (ATCC 35984) (tigr.org). The cell wall sorting motif LPXTG together with C-terminal hydrophobic stretches from the well-characterized LPXTG proteins of S. aureus ClfA, ClfB, FnbpA, FnbpB and Fbe from S. epidermidis are used to find target sequences. Similarity searches are carried out using TBLASTN at tigrblast.tigr.org. The genome sequence of RP62A is imported to the Vector NTI computer software (Informax Inc.), where open reading frames (ORFs) that are at least 50 amino acids in length are identified. The positions of matched sequences in the genome of RP62A that are obtained from the TBLASTN analysis are used to locate selected ORFs. The ORFs are only accepted for further characterization if the LPXTG motif are adjacent to a transmembrane domain followed by a charged C-terminal tail located at the extreme C termini. Identified LPXTG motifs comprising their respective hydrophobic region are used to search for additional target sequences as described above. The presence of signal peptides and prediction of their cleavage sites are analyzed using the SIGNALP (cbs.dtu.dk/services/signalP). Similarity searches to other species at the nucleotide and amino acid level are performed using BLAST (ncbi.nlm.nih.gov). The novel proteins are denoted SesA-SesF as for Staphylococcus epidermidis surface protein A-F.

### Construction and Purification of Recombinant Proteins.

Recombinant protein fragments of identified putative cell surface proteins are expressed and purified using the IMPACT system, New England Biolabs. Gene fragments are amplified by PCR using S. epidermidis strain RP62A genomic DNA as template. Forward and reverse primers are listed in Table 1. The 5′-part of the PCR-products is cleaved with NcoI and the 3′-part of the PCR-products is phosphorylated, following ligation into the pTYB4 vector. This results in a fusion protein of S. epidermidis target protein and the intein/chitin-binding domain (CBD). The target protein constitute the N-terminal part of the fusion and under reducing conditions the intein/CBD part is released by self-cleavage, resulting in a recombinant target protein with only one additional Gly in the C-terminal end. Ligated material is transformed into the E. coli strain ER 2566. Constructs are verified by DNA sequencing using Dyenamic ET Terminator Cycle sequencing Premix Kit and a model 377 Perklin Elmer DNA sequencer. Expression and purification is performed according the manufacturers instructions. Samples of the purified recombinant proteins are analyzed under reducing conditions on an 8-25% gradient SDS-PAGE gel using the PhastSystem (Amersham Biosciences). GST-Fbe is Glutatione-S-Transferase fused to Fbe, produced as previously described.

Occurrence of LPXTG-Containing Genes in Strains of S. epidermidis.

The presence of the putative cell wall associated proteins is examined in 10 isolates recovered from individuals with invasive S. epidermidis infection (5 peritonitis and 5 sepsis strains) and 9 isolates recovered from healthy donors. Lab strain 19 is also included. Genomic DNA is prepared from these strains using the QIA amp Tissue kit obtained from QIAGEN (Hilden, Germany) with the modification that lysostaphin is used in solution one. The presence of the genes is detected by PCR using the corresponding forward primers and reverse primers shown in Table 1.

TABLE 1

Primers used for cloning and PCR of cell wall surface
anchored proteins of S. epidermidis RP62A.

| | |
|---|---|
| Recombinant protein | Primer sequence |

| | |
|---|---|
| Bhp | Forward 5′-CTTAACCATGGCTGAAACGGACAATATCGT |
| | (SEQ ID NO: 1) |
| | Reverse 5′-TTTATTGCTTATTGCTTTTACTGC |
| | (SEQ ID NO: 2) |
| SesA | Forward 5′-CTTAACCATGGATAGTGACAAACATGAAAT |
| | (SEQ ID NO: 3) |
| | Reverse 5′-AGTTTTATCATTAGCCCCTAC |
| | (SEQ ID NO: 4) |
| SesB | Forward 5′-CTTAACCATGGAAGAATTCGAAAGAGTAACTAC |
| | (SEQ ID NO: 5) |
| | Reverse 5′-TTCAGTATCATTTTTAACATCTGT |
| | (SEQ ID NO: 6) |
| SesC | Forward 5′-CTTAACCATGGATGAAAATACTGCAAACCAA |
| | (SEQ ID NO: 7) |
| | Reverse 5′-TTTATCTTGTACATCATTGTTATTAT |
| | (SEQ ID NO: 8) |
| SesD | Forward 5′-CTTAACCATGGGCTGATTATGTAAATGACTCAAAT |
| | (SEQ ID NO: 9) |
| | Reverse 5′-AGCTTTTGTTGTTTGAGCTTC |
| | (SEQ ID NO: 10) |
| SesE | Forward 5′-CTTAACCATGGATTCAGAAAGTACATCATCAAAT |
| | (SEQ ID NO: 11) |
| | Reverse 5′-TGAGATATTACCATCGTCGTC |
| | (SEQ ID NO: 12) |
| SesF | Forward 5′-CTTAACCATGGCCGAAGTAACATCTCATGAT |
| | (SEQ ID NO: 13) |
| | Reverse 5′-CATATCAGATGAACTTTGTTGTGGT |
| | (SEQ ID NO: 14) |

### Antibody Production and Evaluation.

Female 5-6-week-old Wistar rats, weighing approximately 180 g are used. The animals are kept under standard laboratory conditions according to Swedish regulations (Ethical approval No 139-00, Stockholms Södra Djurförsöksetiska Nämnd Huddinge Tingsrätt). Rats are immunized subcutaneously (s.c.) with 20 μg purified protein on days 0 (in Freund's complete adjuvans), 14 and 28 (in Freund's incomplete adjuvans). For all immunizations, the antigens are suspended to a total of 0.5 ml PBS together with adjuvans. Immune sera are collected and analyzed by ELISA

All ELISAs are performed on 96-well, flat-bottomed polystyrene plates (Costar). The titers of antibodies directed against SesA-F, Fbe and Bhp are determined by coating plates with the recombinant proteins (10 μg/ml). Wells are blocked for 1 h at 37° C. with 2% (w/v) BSA in PBS, washed with PBST (PBS+0.05% Tween 20), and serial dilutions of sera in PBST are added. The plates are incubated for 1 h at 37° C., and are washed again with PBST. HRP conjugated rabbit anti-rat IgG (Dako) diluted 1:1000 in PBST is added and incubated for 1 h at 37° C. Plates are washed and developed with TMB (Dako) according to manufacturers instructions.

To determine the total IgG content in sera, plates are coated overnight at room temperature with serial dilutions of serum samples, blocked for 1 h at 37° C. with 2% (w/v) bovine serum albumin (BSA, Sigma) in PBS and washed with PBST. Horseradish peroxidase (HRP) conjugated rabbit anti-rat IgG (Dako) diluted 1:1000 in PBST is added and incubated 1 h at 37° C. Plates are washed and developed with OPD tablets according to the manufacturer's instruction (Dako).

Three commercial IgG preparations, pools 1, 2, and 3, respectively intended for intravenous immunoglobulin (IVIG) therapy are tested in ELISA for reactivity against the Ses proteins as described above, except that the conjugated antibodies are rabbit anti human used at 1000× dilution (Dako). The absorbance values using IgG concentrations at 15 μg/ml are determined. Background values, reactivity against BSA, are subtracted.

Human antibodies specifically directed against Fbe are enriched by affinity chromatography on Fbe-Sepharose. Fbe (10 mg) is coupled to 2 ml CNBr activated Sepharose fast Flow 4C as recommended by the supplier (Pharmacia-Amersham, Uppsala, Sweden) and 25 mg IgG (pool 1, Gamimune N from Bayer) is applied to the column. It is then washed with 35 ml PBS. Finally, the bound IgG is eluted with 0.7% acetic acid with 0.5 M NaCl and dialyzed against PBS. Effluent material is run once more through the Fbe-Sepharose, to remove all anti Fbe-antibodies, to be used a negative control.

### Confirmation of Cell Wall Association.

S. epidermidis RP62A are grown to exponential phase in tryptic soy broth (TSB), reinoculated and further grown 2, 4 or 24 hrs at 37° C. with aeration. Cell wall fractions of the cultures are purified. Briefly, bacterial pellets are resuspended in a buffer with high sugar content (30% raffinose) and lysostaphin 200 μg/ml (DAKO) in presence of a protease inhibitor (mini Complete, Roche Molecular Biochemicals). Protoplasts are excluded by centrifugation, and the supernatants are taken as cell wall fractions. The samples are dialyzed against PBS and precipitated with 70% acetone. The pellets are resuspended in a minimum of PBS. Protein concentrations of the resuspended precipitates are determined by DC Protein Assay (BioRad).

Samples of cell-wall fractions are boiled for 5 min in final electrophoresis sample buffer; 0.125 M Tris/HCl, 4% (w/v) SDS, 20% (v/v) glycerol, 10% (v/v) β-mercaptoethanol, 0.002% (w/v) bromophenol blue. The samples are analyzed in SDS-PAGE (Phast system) with 8-25% acrylamide gels (Amersham Biosciences). Gels are either stained with coomassie blue or passively transferred to nitrocellulose membranes over night. Membranes are blocked with 1% Tween (Merck) in PBS for 20 min at room temperature. Rat antibodies directed against the putative cell wall associated proteins are used at a dilution of 1:500. HRP-conjugated rabbit anti-rat antibodies are used at a dilution of 1:1000 (DAKO). The antibodies are diluted in PBST (0.05% Tween in PBS). Bound antibodies are visualized using 4-chloro-1-naphthol according to manufacturers instructions (Sigma).

### Phagocytosis of Opsonised Bacteria.

A cell line of human monocytes, THP-1 (ATCC TIB-202) is maintained in a 75 cm² flask culture with 24 ml culture medium consisting of 90% RPMI 1640 and 10% fetal bovine serum supplemented with 2 mM L-glutamine (Gibco Invitrogen). Fresh culture medium is supplied three times a week. Cells are centrifuged at 125 g for 10 minutes and resuspended in culture media to a concentration of 10⁶ cells/ml. The media are supplemented with PMA (Sigma) at a final concentration of 100 ng/ml to transform monocytes into macrophages. A 24-well cell culture plate (Costar) is filled with 1 ml of the cell suspension/well and grown at 37 with 5% CO₂. The medium is replaced with fresh culture medium.

S. epidermidis RP62A is grown to exponential phase in tryptic soy broth (TSB), reinoculated and further grown for 4 hrs at 37° C. with aeration. The culture is centrifuged at 7000 g for 15 min and the pellet resuspended in PBS to OD550=1. The samples are kept at −70° C. until use.

S. epidermidis RP62A is thawed and diluted to approximately 10⁷ CFU/ml in PBS. Heat-inactivated (56° C. 30 min.) normal sera or sera from immunized rats are added to a concentration of 10% (v/v). After 1 h of incubating sera/bacteria, 100 μl of the suspension is added to each well with macrophages, giving 1×10⁶ CFU opsonised bacteria/well and a MOI of 1. In some experiments a ten fold higher bacterial concentration is used. The plates are incubated for 90 min, the medium is then replaced with culture medium with 25 μg/ml lysostaphin (Sigma). The plates are further incubated 20 min. at 37° C. and then washed. The supernatants are removed and replaced with 0.1 ml/well Trypsin-EDTA (Sigma) to detach cells from the plastic wells. Nine hundred μl/well sterile water is added to lyse the macrophages. The wells are scraped and the contents are plated onto blood agar plates in serial dilutions. Plates axe incubated at 37° C. over night and colony forming units are counted. When human IgG is used to opsonise, the same procedure is followed and the IgG concentration is 10 μg/ml in the opsonisation step.

### Statistics.

Data from individual opsonisation assays are compared and adjusted in relation to normal serum controls for experiments with rat antibodies and in relation to effluent antibodies or pool-antibodies for experiments with human antibodies. The adjusted data are analyzed with one-way Anova with JMP-IN (SAS institute).

### Results

### Identification of Cell Wall Surface Anchored Proteins.

Ten sequences are identified that contained the signal sequence in the N-terminal and the cell wall localization signal in the C-terminal. Four of those are Fbe, Aap, a cell division protein belonging to the FtsW/RodA/SpoVE family and a protein called Bhp. Bhp is claimed to be related to the as yet uncharacterized Biofilm associated protein Bap in Staphylococcus aureus and to be involved in biofilm formation. Six novel sequences are identified, here denoted SesA-F. SesB shared high identity with Lipoprotein VsaC from S. epidermidis 12228 (Table 2). All of the proteins are denoted “cell wall surface anchor protein family” as published at ncbi.nlm.nih.gov January 2005. There is no published data about the function of these proteins in S. epidermidis.

TABLE 2
Length, NCBI accession number and identities of cell wall
surface anchored proteins of S. epidermidis RP62A.

| | Amino | NCBI | |
|---|---|---|---|
| Protein | acids | accession No. | Identities/similarities (examples of)^{a} |
| Bhp | 2403 | AAW53225 | |
| | | AY028618 | |
| SesA | 1973 | AAW54798 | |
| SesB | 824 | AAW54085 | S. epidermidis ATCC 12228, Lipoprotein VsaC, |
| | | | (AAO04425) |
| SesC | 357 | AAW54808 | S. epidermidis ATCC 12228, Hypothetical protein |
| | | | SE1628 (NP_765183) |
| | | | S. epidermidis strain SR1, Genomic sequence |
| SesD | 237 | AAW54982 | |
| SesE | 676 | AAW53125 | S. epidermidis ATCC 12228, Hypothetical protein |
| | | | SE2232 (NP_765787) |
| | | | S. aureus, Putative surface anchored proteins |
| | | | (YP_044646, NP_375765.1, AAW38666.1, |
| | | | YP_042067.1) |
| | | | S. epidermidis strain SR1, Genomic sequence |
| SesF | 196 | AAW53084 | S. epidermidis ATCC 12228, Hypothetical protein |
| | | | SE2152 (NP_765707.1) |
| | | | S. epidermidis strain SR1, Genomic sequence |
| Fbe/SdrG | 931 | AAW53571 | S. epidermidis Ser-Asp rich fibrinogen binding bone |
| | | AAF72510 | sialoprotein-binding protein (AAO03928) |
| | | | S. epidermidis fibrinogen-binding protein (CAA76638) |
| | | | S. aureus bone sialoprotein-binding protein |
| | | | (CAG39588, CAG42296, BAB94383, BAB41752, |
| | | | BAB56725, CAB75732) |
| | | | S. aureus SdrE (AAW37719, CAA06652) |
| | | | S. aureus SdrD (AAW37718, CAA06651) |
| | | | S. aureus SdrC (AAW37717, CAA06650) |
| | | | Staphylococcus saprophyticus SdrI (AAM90673) |
| | | | S. epidermidis SdrF (AAF72509) |

Note:
^{a}Highly significant (score >200) identities found at http://www.ncbi.nlm.nih.gov/BLAST/using Blastn and Blastx. NCBI accession number in parenthesis.

The proposed cell wall associated protein SdrH is present in RP62A, but is not found with our search criteria due to a short hydrophobic stretch. SdrH is not found in the cell wall fraction. The protein SdrF is truncated in RP62A and is thus not included.

### PCR-Screening in Clinical Isolates.

To determine the presence of the cell wall associated proteins in other strains than the parent strain RP62A, PCR screening of the genomes of 20 other isolates are performed. The DNA is analyzed using primers specific for each gene (Table 1). From this limited collection it appears that Bhp and SesA are less prevalent than the other genes, which are present in almost all isolates. There is no difference in the incidence between isolates derived from healthy patients and isolates derived from sepsis or peritonitis patients (Table 3).

TABLE 3
PCR indicated presence of genes encoding cell wall
surface anchored proteins of S. epidermidis RP62A in
clinical isolates and the laboratory strain 19.

| | | | Peritonitis | Laboratory |
|---|---|---|---|---|
| | Skin isolates | Sepsis isolates | isolates | strain #19 |
| Gene | (n = 9) | (n = 5) | (n = 5) | (n = 1) |
| Bhp | 2/9 | 0/5 | 1/5 | No |
| SesA | 3/9 | 2/5 | 4/5 | No |
| SesB | 9/9 | 5/5 | 5/5 | Yes |
| SesC | 9/9 | 5/5 | 5/5 | Yes |
| SesD | 8/9 | 4/5 | 5/5 | Yes |
| SesE | 9/9 | 5/5 | 5/5 | Yes |
| SesF | 9/9 | 5/5 | 5/5 | Yes |
| Fbe/SdrG | 9/9 | 5/5 | 5/5 | Yes |

Confirmation of Cell Wall Association.

All the immunized animals produced antibodies to the injected recombinant proteins (data not shown). The antibody titers are generally high, with the exception of SesB, which showed poor immunogenic properties. In western blot on cell wall extracts, all the proteins except SesF are identified either as fill-length bands or fragments (data not shown). This indicates that Fbe/SdrG, Blip and SesA-E are expressed by the bacteria, and are bound to the cell wall. In the case of SesF, the inventors are not able to determine any cell wall localization with this method. Control serum did not recognize any of the proteins or fragments on the western blot (data not shown).

Antibodies Directed Against Fbe and SesD Enhance Phagocytosis of S. epidermidis RP62A.

RP62A is a slime producing strain of S. epidermidis. The inventors wanted to see if the cell wall associated proteins identified here, while being expressed on the cell, could be recognized by specific antibodies. Additionally, in the macrophage phagocytosis test used for this purpose, the importance of the novel proteins as targets for antibody therapy is evaluated. FIG. 1 shows that antibodies directed against Fbe can recognize Fbe on the surface of S. epidermidis confirming previous findings. Phagocytosis is also increased by opsonisation with anti SesD serum. None of the antibodies directed against any of the other novel cell wall associated proteins are able to opsonise the bacteria in a sufficient level to increase phagocytosis by macrophages, as compared to normal sera.

### Analysis of Human Antibodies Against Ses Proteins.

Three different commercial pools of IgG intended for IVIG are tested for reactivity against Ses proteins using ELISA. FIG. 2 shows absorbance values after subtraction of background values. It can be seen that pool 3 has the highest titer against all Ses proteins except SesC, against which no reactivity is detected. All Ses proteins, except SesC, are expressed and exposed to the immune system in the population of donors from which the immunoglobulins are derived.

Antibodies against Fbe are purified from IgG pool 1 (Gamimune) using affinity chromatography with Fbe immobilized onto Sepharose. FIG. 3 shows titration curves of the initial IgG pool, eluted antibodies enriched for IgG against Fbe and, as a negative control, the effluent devoid of anti Fbe antibodies. Anti Fbe antibodies can be enriched by this procedure.

The enriched anti Fbe antibodies are next used to assess their biological function as opsonins in two experiments. In the first experiment, S. epidermidis RP69A is incubated either with the enriched anti Fbe antibodies or the effluent IgG fraction lacking anti-Fbe antibodies (in FIG. 4A). The opsonised bacteria are then added to human cultured macrophages on tissue wells. Uptake of bacteria into macrophages is determined after removal of extra cellular bacteria. A significant difference in uptake is found (p<0.05). The ratio of opsonised bacteria to macrophages is about 10 in this experiment. In the next experiment, (FIG. 4B) the ratio bacteria/macrophages is lowered to 1. Here, enriched anti-Fbe antibodies are compared with the pool of IgG. A more pronounced difference (p=0.015) is found here due to the lower burden of bacteria to macrophages. No difference in uptake of opsonised and non-opsonised bacteria can be seen if too many bacteria are used (data not shown). IgG is used at a concentration of 10 μg/ml for all experiments.

The Staphylococcus epidermidis surface (Ses) A-F proteins described and claimed in the present application are further described in Bowden et al, Identification and preliminary characterization of cell-wall-anchored proteins of Staphylococcus epidermidis, Microbiology (2005), 151, 1453-1464. However, the Ses proteins in Bowden et al are described with a different nomenclature. Accordingly, Table 4 is presented herewith to provide a translation of the nomenclature in the present application to the nomenclature as set forth in Bowden et al.

TABLE 4

| Nomenclature of present Application | Nomenclature of Bowden et al |
|---|---|
| Bhp | Bhp, SesD |
| SesA | SesG |
| SesB | SesE |
| SesC | SesH |
| SesD | SesI |
| SesE | SesC |
| SesF | SesB |

What is claimed is:

1. Isolated Staphylococcus epidermidis surface (Ses) A protein.

2. Isolated Staphylococcus epidermidis surface (Ses) B protein.

3. Isolated Staphylococcus epidermidis surface (Ses) C protein.

4. Isolated Staphylococcus epidermidis surface (Ses) D protein.

5. Isolated Staphylococcus epidermidis surface (Ses) E protein.

6. Isolated Staphylococcus epidermidis surface (Ses) F protein.

7. A vaccine comprising a Staphylococcus epidermidis surface (Ses) protein, wherein the Ses protein is selected from the group consisting of SesA protein, SesB protein, SesC protein, SesD protein, SesE protein or SesF protein.

8. A vaccine comprising DNA encoding Staphylococcus epidermidis surface (Ses) protein, wherein the Ses protein is selected from the group consisting of SesA protein, SesB protein, SesC protein, SesD protein, SesE protein or SesF protein.

9. A monoclonal antibody directed against a Staphylococcus epidermidis surface (Ses) protein, wherein the Ses protein is selected from the group consisting of SesA protein, SesB protein, SesC protein, SesD protein, SesE protein or SesF protein.

10. A polyclonal antibody directed against a Staphylococcus epidermidis surface (Ses) protein, wherein the Ses protein is selected from the group consisting of SesA protein, SesB protein, SesC protein, SesD protein, SesE protein or SesF protein.

11. A method of diagnosing an individual with an infection caused by Staphylococcus epidermidis comprising obtaining a sample from the individual and analyzing the sample for a Staphylococcus epidermidis surface (Ses) protein, wherein the Ses protein comprises SesA protein, SesB protein, SesC protein, SesD protein, SesE protein, SesF protein or a combination thereof.

12. A method of diagnosing an individual with an infection caused by Staphylococcus epidermidis comprising obtaining a sample of immunoglobulin from the individual and analyzing the sample for immunological reactivity against Staphylococcus epidermidis surface (Ses) protein, wherein the Ses protein comprises SesA protein, SesB protein, SesC protein, SesD protein, SesE protein, SesF protein or a combination thereof.

13. A diagnostic device, comprising at least one reagent operable to bind a Staphylococcus epidermidis surface (Ses) protein, wherein the Ses protein comprises SesA protein, SesB protein, SesC protein, SesD protein, SesE protein, SesF protein or a combination thereof.
